(19) United States
(12) Patent Application Publication
(10) Pub. No.: US 2021/0363184 A1
(43) Pub. Date: Nov. 25, 2021

(54) PROCESS FOR PREPARATION OF PURE PLECANATIDE

(71) Applicant: Mylan Laboratories Limited, Hyderabad (IN)

(72) Inventors: Ananda KUPPANNA, Hyderabad (IN); Sureshbabu JAYACHANDRA, Hyderabad (IN); Anil Kumar TRIPATHI, Hyderabad (IN); Chandrashekhar SHANIGARAM, Hyderabad (IN); Bulliraju KAMANA, Hyderabad (IN); Sreelatha VANJIVAKA, Hyderabad (IN)

(73) Assignee: Mylan Laboratories Limited, Hyderabad (IN)

(21) Appl. No.: 17/053,548

(22) PCT Filed: May 6, 2019
(86) PCT No.: PCT/IN2019/050359

§ 371 Date: Nov. 6, 2020

(30) Foreign Application Priority Data
May 7, 2018 (IN) 201841017154

(51) Int. Cl. C07K 7/08; C07K 1/20; C07K 1/06

(52) U.S. Cl. CPC C07K 7/08; C07K 1/061; C07K 1/20

(57) ABSTRACT

The present disclosure provides methods for purified Plecanatide by a two-step purification method, novel intermediates that may be used in the preparation of Plecanatide, and purified Plecanatide compositions.

### CROSS-REFERENCE TO RELATED APPLICATIONS

This application is a US national stage application under 35 U.S.C. § 371 of International Application No. PCT/IN2019/050359, filing date May 6, 2019, which claims the benefit of the earlier filing date of Indian Provisional Patent Application No. IN201841017154 filed on May 7, 2018.

The Sequence Listing associated with this application is filed in electronic format via EFS-Web and is hereby incorporated by reference into the specification in its entirety. The name of the text file containing the Sequence Listing is 9157-2007173_ST25.txt. The size of the text file is 7,765 bytes, and the text file was created on Dec. 8, 2020.

### FIELD OF THE INVENTION

The present invention relates to process for preparation of pure Plecanatide by a two-step purification method. The present invention further relates to an improved process for the preparation of pure Plecanatide using novel intermediates.

### BACKGROUND OF THE INVENTION

Plecanatide is a guanylate cyclase-C (GC-C) agonist which increases intestinal transit and fluid through a buildup of cGMP. Plecanatide (brand name TRULANCE®), is a drug approved by the FDA for the treatment of chronic idiopathic constipation (CIC) and irritable bowel syndrome (IBS). Plecanatide is chemically named as L-asparaginyl-L-alpha-aspartyl-L-alpha-glutamyl-L-cysteinyl-L-alpha-glutamyl-L-leucyl-L-cysteinyl-L-valyl-L-asparaginyl-L-valyl-L-alanyl-L-cysteinyl-L-threonylglycyl-L-cysteinyl-L-Leucine cyclic (4->12),(7->15)-bis(disulfide) and is structurally represented by the following chemical Formula I (SEQ ID NO: 1):

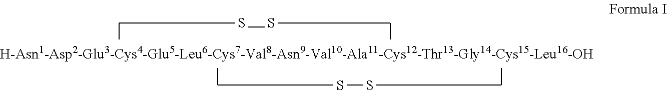

Plecanatide is a 16-amino acid peptide (NDECELCVNVACTGCL, SEQ ID NO: 1) and crystalizes in an amorphous form, as a white to off-white powder. Plecanatide is soluble in water. Commercial formulations of plecanatide, e.g., TRULANCE®, are available as a 3 mg tablet suitable for oral administration.

U.S. Pat. No. 7,041,786 first disclosed Plecanatide and indicated that the Plecantide was synthesized and purified using a published procedure disclosed by Klodt, et al., J peptide Res. 50:222-230 (1997), which disclosed solid-phase synthesis of peptides.

U.S. Pat. No. 9,580,471 discloses a process for the preparation of Plecanatide. The process involved in this patent uses a fully protected Fragment-A Boc-Asn(Trt)-OH having a bulky trityl group, which causes the coupling reaction to become troublesome. Furthermore, cleavage of Protected Plecanatide requires the use of highly toxic, pungent and volatile reagents, e.g., 1,2-ethanedithiol (EDT). Also, after dicyclization, the crude Plecanatide is purified using polystyrene adsorbent material which generates a large amount of waste.

The inventors of the present invention developed an improved process for the preparation of pure Plecanatide, which minimizing or avoiding the use of highly toxic, pungent and/or volatile reagents required by previous methods, e.g. 1,2-ethanedithiol (EDT) and polystyrene adsorbent material.

### SUMMARY OF THE INVENTION

In some aspects, the present disclosure provides methods for producing and/or purifying Plecanatide, as well purified Plecanatide produced using such methods.

One aspect of the present invention provides a process for the preparation of pure Plecanatide comprising the following steps:

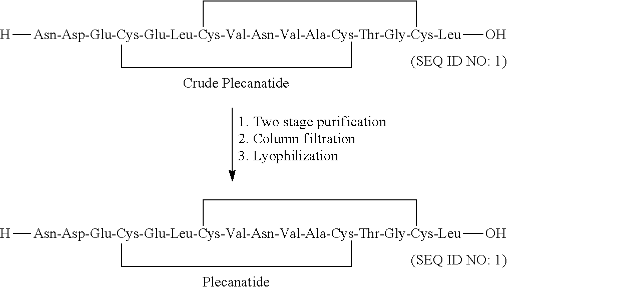

In this aspect, the oxidized crude peptide solution pH may be adjusted to 4-5 with acetic acid before injecting into preparative HPLC. The first stage of purification is performed using reverse phase preparative HPLC by using a 50 mm DAC Column packed with C18 RP silica as a stationary phase. Mobile Phase A may be ammonium acetate solution in water and acetic acid, and Mobile Phase B may be acetonitrile and Mobile phase A. The diluted fractions may be purified using Mobile Phase A, acetic acid in water, and Mobile Phase B, methanol. The product may be eluted with 50% Mobile Phase B/Mobile Phase A to obtain the required Plecanatide fractions. Further, the main pooled fractions are diluted with 20% water and repurified by using 0.4% aq. acetic acid and methanol. These fractions may be pooled and diluted with 20% water and subjected to a second stage of purification by reverse phase preparative HPLC using a 50 mm DAC column packed with C18 silica as a stationary phase and a mobile phase consisting of aqueous ammonium bicarbonate/acetonitrile mixture at pH of 7 to obtain >98% pure Plecanatide.

The pure eluted fractions are concentrated using Tangential flow filtration (TFF)/nano filtration method and lyophilized to obtain pure Plecanatide.

Another aspect of the present invention provides a partially protected Fragment A of compound of Formula II (SEQ ID NO: 2).

Boc-Asn-Asp(OtBu)-Glu(OtBu)-Cys(Trt)-Glu(OtBu)-Leu-OH Formula II.

Yet another aspect of the present invention provides a protected linear Plecanatide of compound of the Formula III (SEQ ID NO: 3).

Boc-Asn-Asp(OtBu)-Glu(OtBu)-Cys(Trt)-Glu(OtBu)-Leu-Cys(Acm)-Val-Asn(Trt)-Val-Ala-Cys(Trt)-Thr(tBu)-Gly-Cys(Acm)-Leu-OtBu. Formula III.

### DETAILED DESCRIPTION OF THE INVENTION

The present invention relates to a process for the preparation of pure Plecanatide by a two-step purification method. Further, the present invention relates to an improved process for the preparation of pure Plecanatide using novel intermediates.

In one exemplary aspect, the present invention relates to a process for the preparation of pure Plecanatide comprising the steps of:

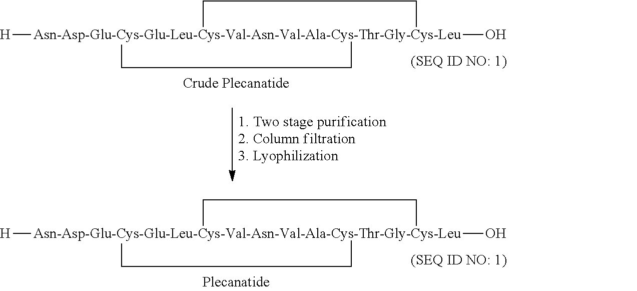

In this aspect, the oxidized crude peptide solution pH may be adjusted to 4-5 with acetic acid before injecting into preparative HPLC. The first stage purification is completed by reverse phase preparative HPLC by using a 50 mm DAC Column packed with C18 silica as a stationary phase, with a Mobile Phase A (0.05M ammonium acetate solution in water and acetic acid), and Mobile Phase B (75% Acetonitrile and 25% Mobile phase A). Further, the main pooled fractions are diluted with 20% water and repurified using 0.4% aq. acetic acid and methanol. Then, the main pooled fractions obtained from above are diluted with 20% water, and the fractions are subjected to a second stage of purification by reverse phase preparative HPLC using a 50 mm DAC column packed with C18 silica as a stationary phase and a mobile phase consisting of 0.005M aqueous ammonium bicarbonate and acetonitrile mixture at pH 7 to obtain more than 98% pure Plecanatide. The pooled fractions are concentrated using 300 Dalton membrane filtration to get about 70-80% by volume of concentrated mass. The obtained mass is lyophilized to obtain Plecanatide (Purity >98%).

Yet another embodiment of the present invention provides a process for the preparation of pure Plecanatide comprising the steps of:

### Stage-I: Preparation of Fragment-A

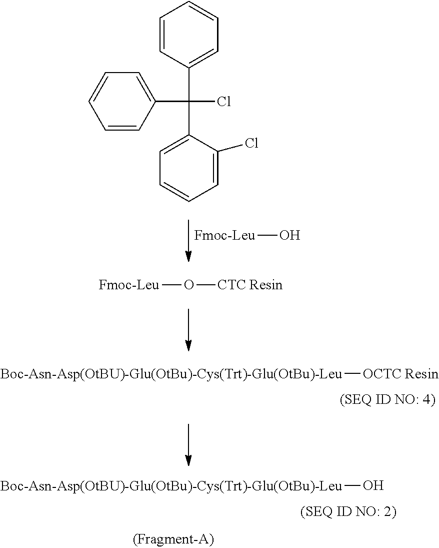

At this stage, dichloromethane (DCM) is added to 2-chlorotrityl resin, and the resin is allowed to swell. The first amino acid Fmoc-Leu-OH is dissolved in dichloromethane and cooled. To this cooled solution, diisopropylethylamine is added and maintained. This cooled solution is added to the above resin material, the solvent is removed by filtration, and the resin is washed with N,N-dimethyl formamide (DMF) followed by dichloromethane. The resin is placed in a peptide synthesizer and dichloromethane, methanol and diisopropylethylamine are added and maintained. The solvent is drained and the resin washed with N,N-dimethyl formamide followed by dichloromethane. Deprotection of Fmoc is performed by addition of 20% piperidine in N,N-dimethyl formamide. The solvent is drained monitoring the progress of the reaction by Kaiser Test. After completion of the reaction, the resin is washed with N,N-dimethyl formamide followed by dichloromethane. The next coupling of the amino acid Fmoc-Glu(OtBu)-OH is performed using a coupling reagent, hydroxybenzotriazole (HOBT), and diisopropylcarbodimide in the presence of N,N-dimethyl formamide followed by washing the resin with N,N-dimethyl formamide. Continuing the deprotection process with 20% piperidine in N,N-dimethyl formamide to the sequence of the next four amino acids Fmoc-Cys(Trt)-OH, Fmoc-Glu(OtBu)-OH, Fmoc-Asp(OtBu)-OH and Boc-Asn-OH using N,N-dimethyl formamide as a solvent and hydroxybenzotriazole and N,N-Diisopropylcarbodiimide. After completion of synthesis, the resin is washed with methanol followed by methyl tert-butyl ether (MTBE). Finally, the resin containing the peptide (SEQ ID NO: 4) is dried under vacuum tray dryer. The resin is added to a cooled solution containing triflouroacetic acid and dichloromethane and filtered followed by neutralization to pH 6 to 7 using diisopropylethylamine (DIPEA). This step is repeated three times. The combined mother liquor is concentrated and methanol is added to the obtained residue to form a slurry. The slurry is added to a cooled solution of water and the solid obtained is separated by filtration and washed with water followed by n-heptane and dried to obtain partially protected Fragment-A (SEQ ID NO: 2).

### Stage-II: Preparation of Fragment-B

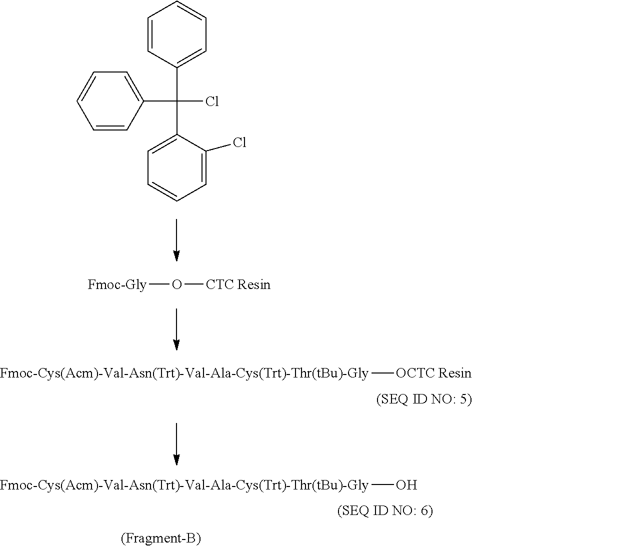

An amino acid Fmoc-Gly-OH loaded chlorotrityl chloride (CTC) resin may be prepared which is dissolved in an inert solvent such as dichloromethane, and the reaction is performed in the presence of a base selected from the group consisting of diisopropylethylamine, triethylamine, sym-collidine, 4-dimethyl amino pyridine, and pyridine, preferably diisopropylethylamine, followed by deprotection of Fmoc-Gly-O-CTC resin performed by using 20% piperidine solution using a suitable solvent like N,N-dimethyl formamide. Continue the coupling of next amino acid Fmoc-Thr(OtBu)-OH by using coupling reagents selected from the group consisting of dicyclohexyl carbodiimides, diisopropyl carbodiimides, 1-ethyl-3-(3′-dimethylaminopropyl) carbodiimides, preferably diisopropylcarbodimide and a coupling additive selected from the group consisting of 1-hydroxybenzotriazole (HOB T), 1-hydroxy-6-chloro benzotriazole, 1-hydroxy-7-azabenzotriazole (HOAT), ethyl cyano(hydroxyamino)acetate (Oxymea pure), preferably 1-hydroxybenzotriazole (HOBT) and solvent selected from N,N-dimethyl formamide (DMF), N-methyl pyrrolidone (NMP) preferably N,N-dimethyl formamide (DMF), followed by deprotection using 20% piperidine in N,N-dimethyl formamide. The same process is repeated for the next four amino acids Fmoc-Cys(Trt)-OH, Fmoc-Ala-OH, Fmoc-Val-OH and Fmoc-Cys(Acm)-OH, and the obtained resin (SEQ ID NO: 5) is cleaved using 1% trifluoro acetic acid solution in dichloromethane as a solvent to obtain protected Fragment-B (SEQ ID NO: 6).

### Stage-III: Preparation of Protected Fragment-C

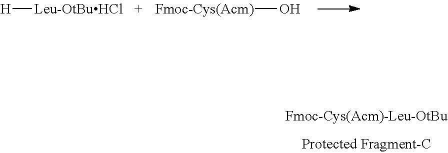

Protected Fragment C may be obtained by the coupling of two amino acids Fmoc-Cys(Acm)-OH and H-Leu-OtBu.HCl in the presence of a coupling reagent selected from the group consisting of HOBt/HBTU, HOBt/TBTU, HOAt/HATU, HOOBt/DEPBT, and Cl-HOBt/PyBoP, and a base selected from the group consisting of diisopropylethylamine, triethylamine, sym-collidine, 4-dimethyl amino pyridine, and pyridine, preferably diisopropylethylamine. The solvent is selected from the group N,N-dimethyl formamide (DMF) and N-methyl pyrrolidone (NMP), preferably DMF to get Protected Fragment-C.

### Stage-IV: Preparation of Protected Fragment-(B+C)

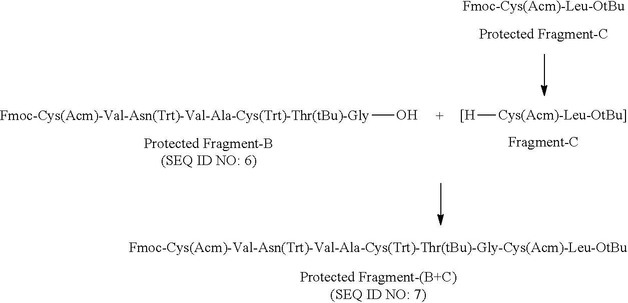

At this stage, Protected Fragment-C may be deprotected with piperidine in presence of dichloromethane which is coupled with Protected Fragment-B (SEQ ID NO: 6) in the presence of a coupling reagent selected from the group consisting of hydroxybenzotriazole (HOBT), (2-(1H-benzotriazol-1-yl)-1,1,3,3-tetramethyluronium hexafluorophosphate (HBTU), TBTU/HOBt, HATU/HOAt, DEPBT/HOOBt, PyBoP/C1-HOBt and diisopropylethylamine, triethylamine, sym-collidine, 4-dimethyl amino pyridine, and pyridine, preferably diisopropylethylamine in the presence of N,N-dimethyl formamide to obtain the Protected Fragment-(B+C) (SEQ ID NO: 7).

### Stage-V: Preparation of Fragment-(B+C)

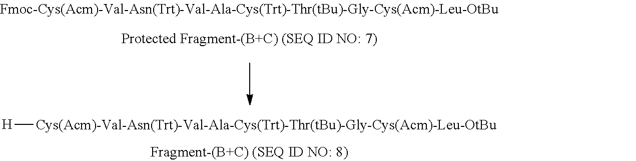

According to the present invention, Fragment-(B+C) (SEQ ID NO: 8) is obtained by deprotection of Protected Fragment-(B+C) (SEQ ID NO: 7) using deprotecting agents 4-methylpiperidine or piperazine and piperidine or a non-nucleophilic base, e.g., DBU in a solvent N, N-dimethylformamide.

### Stage-VI: Preparation of Protected Plecanatide

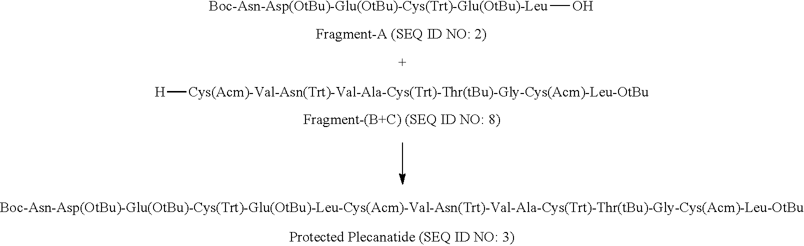

According to the present invention, Protected Plecanatide (SEQ ID NO: 3) is obtained by coupling partially protected Fragment-A (SEQ ID NO: 2) with Fragment-(B+C) (SEQ ID NO: 8) using a coupling reagent selected from the group consisting of HOBt/HBTU, HOBt/TBTU, HOAt/HATU, HOOBt/DEPBT, and Cl-HOBt/PyBoP; and a base selected from the group consisting of diisopropylethylamine, triethylamine, sym-collidine, 4-dimethyl amino pyridine, and pyridine, preferably diisopropylethylamine. The solvent is selected from the group N, N-dimethyl formamide (DMF), and N-methyl pyrrolidone (NMP), preferably DMF as the solvent to obtain the Protected Plecanatide.

### Stage-VII: Preparation of Linear Plecanatide

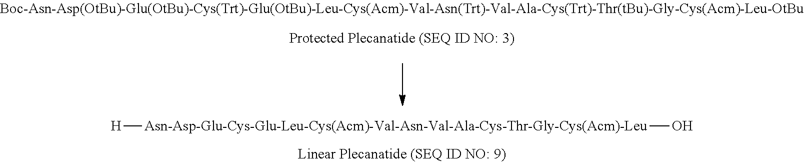

According to the present invention, Protected Plecanatide (SEQ ID NO: 3) is cleaved by using reducing or deprotecting agents like triisopropyl silane (TIPS) with trifluoro acetic acid in the presence of dithiothreitol (DTT) to obtain Linear Plecanatide (SEQ ID NO: 9).

### Stage-VIII: Preparation of Plecanatide

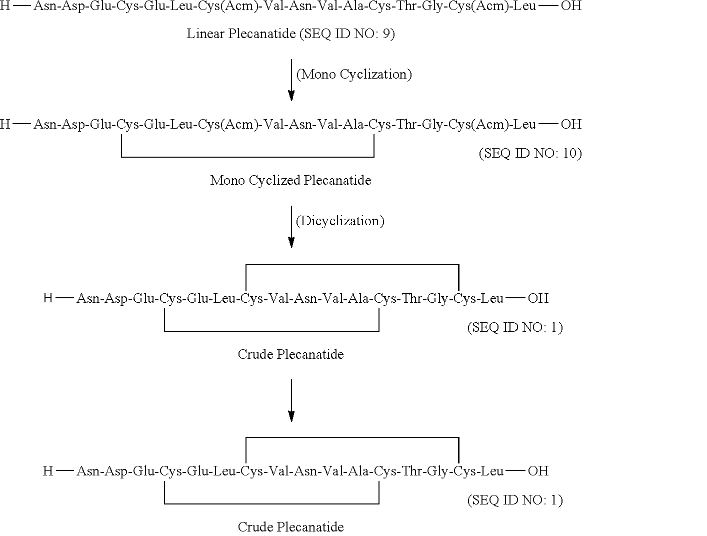

According to the present invention, Linear Plecanatide (SEQ ID NO: 9) is dissolved with purified water/acetonitrile mixture in presence of aq. ammonia to adjust the pH of the reaction mass to about 8 to 9 followed by addition of 3.5% of hydrogen peroxide. After completion of monocyclization (SEQ ID NO: 10), the pH of the reaction mass is adjusted to about 3 to 4 by addition of acetic acid and iodine solution in acetonitrile to get Crude Plecanatide (SEQ ID NO: 1). The formation of dicyclization is monitored by HPLC. After completion of the reaction, the pH of reaction mass is adjusted to between 6 to 7 by addition of aq. ammonia solution, followed by addition of Amberjet 9000-OH resin to remove the excess of iodine. The Crude Plecanatide solution is purified as described above to obtain pure Plecanatide having a purity of more than 98%.

In yet another exemplary aspect, the disclosure provides a Partially-Protected Fragment A of the compound of Formula II (SEQ ID NO: 2).

Boc-Asn-Asp(OtBU)-Glu(OtBu)-Cys(Trt)-Glu(OtBu)-Leu-OH Formula II.

In yet another exemplary aspect, the present disclosure provides a protected Linear Plecanatide of the compound of Formula III (SEQ ID NO: 3).

Boc-Asn-Asp(OtBu)-Glu(OtBu)-Cys(Trt)-Glu(OtBu)-Leu-Cys(Acm)-Val-Asn(Trt)-Val-Ala-Cys(Trt)-Thr(tBu)-Gly-Cys(Acm)-Leu-OtBu. Formula III.

Table 1 below summarizes the two-step purification method with a purity and a mobile phase solvent system.

TABLE 1

| Stages of | | |
|---|---|---|
| purification | Purity Level | Mobile phase solvent system |
| First stage of | >95% | aq. ammonium acetate/acetonitrile |
| purification | | |
| Second stage | >98% | aq. ammonium bicarbonate/acetonitrile |
| of purification | | |

The purified plecanatide prepared according to the present invention is substantially free of one or more of the following impurities. In still further aspects of the disclosure, a purified plecanatide may contain undetectable levels of one or more of the following impurities.

### Topoisomer Impurities (SEQ ID NO: 11):

L-Leucine, L-asparaginyl-L-alpha-aspartyl-L-alpha-glutamyl-L-cysteinyl-L-alpha-glutamyl-L-leucyl-L-cysteinyl-L-valyl-L-asparaginyl-L-valyl-L-alanyl-L-cysteinyl-L-threonylglycyl-L-cysteinyl-, Cyclic (4→12),(7→15)-bis(disulfide)
(or)

H-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶-OH, Cyclic (4→12),(7→15)-bis(disulfide)

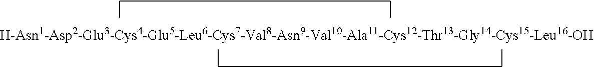

### 9-Asp-Plecanatide (SEQ ID NO: 12):

L-Leucine, L-asparaginyl-L-alpha-aspartyl-L-alpha-glutamyl-L-cysteinyl-L-alpha-glutamyl-L-leucyl-L-cysteinyl-L-valyl-L-aspartyl-L-valyl-L-alanyl-L-cysteinyl-L-threonylglycyl-Lcysteinyl-, Cyclic (4→12),(7→15)-bis(disulfide)
(or)

H-Asn¹-Asp²-Glu³-Cys⁴-Glu⁵-Leu⁶-Cys⁷-Val⁸-Asp⁹-Val¹⁰-Ala¹¹-Cys¹²-Thr¹³-Gly¹⁴-Cys¹⁵-Leu¹⁶-OH, Cyclic (4→12),(7→15)-bis(disulfide)

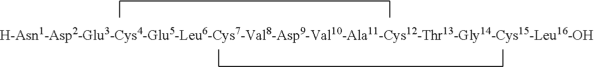

### 2-Iso-Asp-Plecanatide (SEQ ID NO: 13):

L-Leucine, L-asparaginyl-L-alpha-iso-aspartyl-L-alpha-glutamyl-L-cysteinyl-L-alpha-glutamyl-L-leucyl-L-cysteinyl-L-valyl-L-asparaginyl-L-valyl-L-alanyl-L-cysteinyl-L-threonylglycyl-L-cysteinyl-, Cyclic (4→12),(7→15)-bis(disulfide)
(or)

H-Asn¹-Iso-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶-OH, Cyclic (4→12),(7→15)-bis(disulfide)

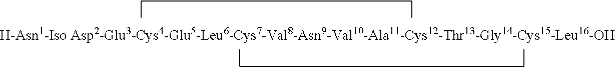

### Plecanatide Isomer-I (SEQ ID NO: 14):

L-Leucine, L-asparaginyl-L-alpha-aspartyl-L-alpha-glutamyl-L-cysteinyl-L-alpha-glutamyl-L-leucyl-L-cysteinyl-L-valyl-L-asparaginyl-L-valyl-L-alanyl-L-cysteinyl-L-threonylglycyl-L-cysteinyl-, Cyclic (4→7),(12→15)-bis(disulfide)
(or)

H-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶-OH, Cyclic (4→7),(12→15)-bis(disulfide)

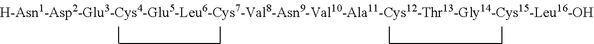

### Plecanatide Isomer-II (SEQ ID NO: 15):

L-Leucine, L-asparaginyl-L-alpha-aspartyl-L-alpha-glutamyl-L-cysteinyl-L-alpha-glutamyl-L-leucyl-L-cysteinyl-L-valyl-L-asparaginyl-L-valyl-L-alanyl-L-cysteinyl-L-threonylglycyl-L-cysteinyl-, Cyclic (4→15),(7→12)-bis(disulfide)
(or)

H-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶-OH, Cyclic (4→15),(7→12)-bis(disulfide)

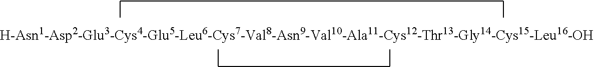

The following examples are provided to illustrate processes in accordance with the present disclosure. They, are however, not intended to limit the scope of the present invention in any way and several variants of these examples would be evident to a person ordinarily skilled in the art.

### Example 1: Preparation of Fragment-A

In a clean dry peptide synthesizer vessel charged with 200.0 g of 2-Chlorotrityl resin add 2000 ml of dichloromethane and allow the resin for to swell for 1 hour. Drain the solvent. The first amino acid Fmoc-Leu-OH (158.0 gm, 2.0 eq.) is dissolved in dichloromethane (1000 ml) and cooled to 5-10° C. In the cooled solution, add diisopropylethylamine (234 ml) and stirred the solution for 5 minutes. Place the cooled solution into the peptide synthesizer vessel and stir for 5 hrs. at a temperature of 20-25° C. The solvent is removed by filtration. The resin is washed with N,N-dimethyl formamide (2×1000 ml) followed by dichloromethane (2×1000 ml). In the peptide synthesizer vessel containing the resin, charge a solution of 1020 ml of dichloromethane, 120 ml of methanol, and 400 ml of diisopropylethylamine, and stir the reaction mass for 1 hour at a temperature of 20-25° C. to end cap the resin. Next, drain the solvent and washed the resin with N,N-dimethyl formamide (2×1000 ml) followed by dichloromethane (2×1000 ml). Deprotection of Fmoc is performed by adding cooled solution of 1000 ml of 20% piperidine in N,N-dimethyl formamide and stirring the mass for 15 minutes. The solvent is drained, cooled solution of 1000 ml of 20% piperidine in N,N-dimethyl formamide is added, and the mass is stirred for 15 minutes. The progress of the reaction is monitored by Kaiser Test. After completion of reaction, wash the resin with N,N-dimethyl formamide (2×1000 ml) followed by dichloromethane (2×1000 ml). Continue the coupling of next amino acid using Fmoc-Glu(OtBu)-OH (136.0 gm, 2.0eq), coupling reagent hydroxybenzotriazole (43.0 gm, 2.0 eq) and diisopropylcarbodimide (50.2 ml, 2.0 eq), the N,N-dimethyl formamide (1000 ml) for 2 hours at a temperature of 20-25° C. Next, wash the resin with N,N-dimethyl formamide (2×1000 ml) followed by dichloromethane (2×1000 ml) and check the Kaiser Test. Continue the deprotection with 20% piperidine in N,N-dimethyl formamide and sequence of the next four amino acids Fmoc-Cys(Trt)-OH (188 gm, 2.0 eq), Fmoc-Glu(OtBu)-OH (136 gm, 2.0 eq), Fmoc-Asp(OtBu)-OH (132.0 gm, 2.0 eq.) and Boc-Asn-OH (74.0 gm, 2.0 eq.), N,N-dimethyl formamide is used as a solvent, and hydroxybenzotriazole and DIC are used as the coupling reagent. After completion of the synthesis, wash the resin with methanol (2×1000 ml) followed by methyl tert.butyl ether (2×1000 ml). Finally, the resin containing the peptide is dried under a vacuum tray dryer for 2 hours at temperatures of 40-45° C. to get resin containing peptide of 440 g. The 35 ml of trifluoro acetic acid and 3500 ml of dichloromethane are charged into a round bottom flask, cooled to 0 to 5° C., and resin is added under cooled conditions and stirring. The mass is stirred for 10 minutes. The reaction mass is filtered, and mother liquor is neutralized (pH 6 to 7) by addition of diisopropylethylamine. Again, 30 ml of trifluoro acetic acid and 3000 ml of dichloromethane are cooled to 0 to 5° C., and resin is added under cooled conditions and stirring. The mass is stirred for 10 min. The reaction mass is filtered, and the mother liquor is neutralized (pH 6 to 7) by addition of diisopropylethylamine. Again, 30 ml of trifluoro acetic acid and 3000 ml of dichloromethane are cooled to 0 to 5° C., and the resin is added under cooled conditions and stirring. The mass is stirred for 10 min. The reaction mass is filtered, and mother liquor is neutralized (pH 6 to 7) by addition of diisopropylethylamine. The combined mother liquor is distilled on rotatory evaporator at a temperature of 45-50° C. The residue is swapped with methanol (300 ml). In the residue mass, 150 ml methanol is added to get a slurry mass. The slurry is added into a cooled solution of water (3000 ml) (temperature of 5 to 10° C.) and stirred for 2 hours. The product is isolated by filtration and the cake is washed with water (2×500 ml) followed by displacement washing with n-heptane (2×500 ml). The cake is sucked dry for 30 minutes. The material is dried in a vacuum tray drier under reduced pressure at 45-50° C. to get 200 g of Fragment-A (SEQ ID NO: 2).

Wt. of Fragment: 200 g

Yield w/w=1.0 (w.r.t. CTC Resin)

### Example 2: Preparation of Fragment-B

In a clean dry peptide synthesizer vessel, charge 100.0 g of 2-Chlorotrityl resin, add 1000 ml of dichloromethane, and allow the resin to swell for 1 hour. Drain the solvent. In peptide synthesizer, charge 600 ml of dichloromethane, 100 ml of thionyl chloride, and 5 ml of N,N-dimethyl formamide at a temperature of 20-25° C., and stirr the mass for 1 hour. Drain the solvent and wash the resin with dichloromethane (3×600 ml), N,N-dimethyl formamide (2×600 ml), and dichloromethane (2×600 ml). Drain the solvent. The first amino acid Fmoc-Gly-OH (94.0 gm, 2.0 eq) is dissolved in dichloromethane (600 ml) and cooled to 5-10° C. In the cooled mass, 80 ml of diisopropylethylamine is added and stirred for 5 minutes. Place the cooled reaction mass into the peptide synthesizer vessel and stir for 5 hours at temperature of 20-25° C. Drain the solvent. The resin attached with first amino acid is washed with N,N-dimethyl formamide (2×600 ml) and dichloromethane (2×600 ml). After washing, add 510 ml of dichloromethane, 60 ml of methanol followed by 30 ml of diisopropylethylamine to the reaction mass and stir for 1 hour to end cap the resin. Next, remove the solvent by filtration. Wash the resin with N,N-dimethyl formamide (2×1000 ml) followed by dichloromethane (2×1000 ml). Deprotection of Fmoc is done with chilled 20% piperidine in N,N-dimethyl formamide (2×600 ml) for 15 min twice. The progress of reaction is monitored by Kaiser Test. Wash the resin with N,N-dimethyl formamide (2×1000 ml) followed by dichloromethane (2×1000 ml). After deprotection of Fmoc group, the coupling of the next amino acid is performed. Fmoc-Thr(tBu)-OH (64.0 gm, 2.0 eq) is dissolved into 600 ml of N,N-dimethyl formamide and the coupling reagent hydroxybenzotriazole (22.0 gm, 2.0 eq) is added. The mass is cooled to a temperature of 0-5° C. To the cooled mass, diisopropyl carbodimide (26.0 ml, 2.0 eq) is added. The cooled solution is charged into the peptide synthesizer vessel and stirred for 2 hours at 20-25° C. The progress of reaction is monitored by Kaiser Test. After completion of reaction, drain the solvent and wash the resin with N,N-dimethyl formamide (2×600 ml) followed by dichloromethane (2×600 ml). Deprotection of Fmoc is performed using chilled 20% piperidine in N,N-dimethyl formamide (2×600 ml). The reaction mass is stirred for 15 minutes, and the solvent is drained. The progress of reaction is monitored by Kaiser Test. Wash the resin with N,N-dimethyl formamide (2×1000 ml) followed by dichloromethane (2×1000 ml). After deprotection of Fmoc group, continue the coupling sequence of the next six amino acids: Fmoc-Cys(Trt)-OH (94.0 gm, 2.0 eq.), Fmoc-Ala-OH (50.0 gm, 2.0 eq.), Fmoc-Val-OH (54.0 gm, 2.0 eq.), Fmoc-Asn(Trt)-OH (96.0 gm, 2.0 eq.), Fmoc-Val-OH (50.0 gm, 2.0 eq.), and Fmoc-Cys(Acm)-OH (66.0 gm, 2.0 eq.) in their order in the presence of N,N-dimethyl formamide as a solvent, and hydroxybenzotriazole and DIC is used as a coupling reagent. After completion of synthesis, wash the resin with methanol (2×600 ml) followed by methyl tert.butyl ether (2×600 ml). Dry the material for 2 hours in a vacuum tray dryer at 40-45° C. to get 250 g of resin containing peptide. In another round bottom flask charged the trifluoro acetic acid (20 ml) and dichloromethane (2000 ml), cool the solution to 0 to 10° C. and add the resin into the cooled solution, then stir for 15 minutes under cooled conditions. Filter the resin, and the mother liquor is neutralized (pH 6 to 7) with diisopropylethylamine. The above operation is repeated three times. The combined mother liquor is distilled on a rotatory evaporator at a temperature of 45-50° C. The residue is swapped with methanol (150 ml). To the residue mass, add 75 ml of methanol to get a slurry mass. The slurry is added into a cooled solution of water (1500 ml) (temperature of 5 to 10° C.) and stirred for 2 hours. The product is isolated by filtration and cake is washed with water (2×250 ml) followed by displacement washing with methyl tertbutyl ether (2×250 ml). Suck dry the cake for 30 minutes. Dry the material in a vacuum tray drier under reduced pressure at 45-50° C. to get 120 g of Fragment-B (SEQ ID NO: 6).

Yield w/w=1.2 (w.r.t. CTC resin)

### Example 3: Preparation of Protected Fragment-C

A solution of Fmoc-Cys(Acm)-OH (277.5 gm, 1.0 eq), H-Leu-OtBu.HCl (150.0 gm, 1.0) and hydroxybenzotriazole (90.6 gm, 1.0) in N,N-dimethyl formamide (1350 ml) is cooled to 5-10° C. Add HBTU (254.12 gm) to the above reaction mass. Next, diisopropylethylamine (302.3 gm) is added dropwise for 2 hours at 5-10° C. and stirred for 3 hours at room temperature. The reaction mixture is diluted with ethyl acetate (1350 ml) and washed with 5% H₃PO₄ (2×1000 ml), 5% aq. sodium bicarbonate (2×800 ml), and 15% sodium chloride (2×800 ml). Evaporate the ethyl acetate and add the oily mass to n-heptane. Stir the thick mass and filter the solid. Dry the product under vacuum at 45-50° C. to get protected Fragment-C (370.0 gm).

Wt. of Fragment: 370 g

Yield w/w=1.33

### Example 4: Preparation of Protected Fragment-(B+C)

Charge dichloromethane (1840 ml) into a 5.0 liter round bottom flask fitted with an overhead stirrer, charge the Protected Fragment-C (230 gm) and stir. Cool the mass to 5-10° C. In the cooled solution, add piperidine (221 ml) dropwise over a period of 30 minutes. After complete addition of the piperidine, raise the temperature of the reaction mass to 20-25° C. and stir for 2 hours. The progress of reaction is monitored by HPLC. When the unreacted Protected Fragment-C is not more than 1%, concentrate the reaction mass to remove the solvent. The concentrated mass is stirred with n-heptane (460 ml), and the solvent is distilled to get a solid mass. In the concentrated mass, n-Heptane (960 ml) is added and stirred for 15 minutes. Decant the supernatant liquid. Then, the residue is dissolved into ethyl acetate (1840 ml) to get a clear solution. The clear solution is washed with a mixture of 2×1150 ml of NaH₂PO₄/Na₂HPO₄ solution (92 gm of NaH₂PO₄ and 92 gm of Na₂HPO₄ dissolved into 2300 ml of water, pH˜6). Finally, the organic layer is washed with 15% sodium chloride solution (2×1150 ml). The layer is then concentrated on a rotatory evaporator at bath temperature of 40-45° C. to get a sticky solid. The solid is further stirred with 2×690 ml of n-heptane, and the supernatant liquid is decanted. Concentrate the mass under vacuum. The oily mass is dissolved into 4500 ml of N,N-dimethyl formamide and charged into a 10 liter round bottom flask while stirring. Add the 480 gm (0.25 mol.) of Fragment-C and 64.6 gm of hydroxybenzotriazole into the flask. Stir the reaction mass to get a clear solution. The reaction mass is cooled to −5 to −10° C. 181.3 gm (0.48 mol.) of HBTU is added followed by drop wise addition of 289 ml of diisopropylethylamine at a temperature of −5 to −10° C. over a period of 1 hour. Next, raise the temperature of the reaction mass to 20-25° C. and maintain the temperature for 2 hours. Add 6.4 g of hydroxybenzotriazole and 18.1 g of HBTU followed by drop wise addition of 39 ml of diisopropylethylamine. Stir the reaction mass for 1 hour further. Again, add 6.4 g of hydroxybenzotriazole and 18.1 g of HBTU followed by drop wise addition of 39 ml of diisopropylethylamine. Stir the reaction mass for 1 hour further. The progress of reaction is monitored by UPLC (The unreacted Fragment-B limit NMT 5%). A mixture of 180 ml of hydrochloric acid (35%) and 2250 ml of water is mixed and the solution is cooled to 0 to 10° C. After completion of reaction, pour the reaction mass into the cooled solution of diluted hydrochloric acid. Stir the reaction mass at a temperature of 5 to 15° C. over a period of 15 minutes. Filter the reaction mass under reduced pressure and wash the cake with water (6750 ml). The cake is further washed with 5% (w/v) sodium bicarbonate solution (6750 ml). The cake is sucked dry and slurry washed with methyl tertbutyl ether 6750 ml and filtered. The cake is further washed with 6750 ml of methyl tertbutyl ether and sucked dry. The cake is dried under reduced pressure at a temperature of 45-50° C.

Wt. of Protected Fragment (B+C) (SEQ ID NO: 7): 540 g

Yield w/w=1.20 (Theory=1.21)

### Example 5: Preparation of Fragment-(B+C)

Protected Fragment-(B+C) (SEQ ID NO: 7) 480 g (0.25 mol.) is dissolved in 4800 ml of N,N-dimethyl formamide at a temperature of 20-25° C., and the mass is cooled to 0-10° C. 960 ml of pieridine is added dropwise over a period of 60 minutes and stir the reaction mass for 60 minutes at 0-10° C. Slowly raise the temperature of the reaction mass to 15-25° C. and stir the reaction mass for 1 hour. The progress of the reaction is monitored by UPLC (the unreacted Fragment-B). After completion of the reaction, the reaction mixture is poured into a mixture of ice (1450 g) and 25 ml hydrochloric acid (35%). Stir the reaction mass for 15 minutes and filter the reaction mass. Wash the cake with 7200 ml of water followed by a displacement wash with 7200 ml of methyl tert.butyl ether. The cake is slurry washed with 7200 ml of methyl tert.butyl ether and filtered. The cake is further washed with 7200 ml of methyl tert.butyl ether. Finally, the cake is dried under reduced pressure at 45-50° C. for 16 hrs.

Wt. of Fragment-(B+C) (SEQ ID NO: 8): 370 g,

Yield w/w=0.77 (Theory=0.89)

### Example 6: Preparation of Protected Plecanatide

Charge 4000 ml of dry N,N-dimethyl formamide into a 10 liter round bottom flask fitted with an overhead stirrer under nitrogen atmosphere. Charge 350 g (0.2 mol.) of Fragment-(B+C) (SEQ ID NO: 8), 275.7 g (0.22 mol.) of Fragment-A (SEQ ID NO: 2) and 48.9 g of hydroxybenzotriazole while stirring. Charge the 1250 ml of N,N-dimethyl formamide and stir the mass for 15 minutes. The reaction mass is cooled to −5 to −10° C. and 175 g (0.46 mol.) of HBTU is charged in the reaction mass. 175 ml of DIEA is added dropwise at a temperature to 0 to −10° C. over a period of 1 hour. Further stir the reaction mass for 1 hour. Slowly raise the temperature of reaction mass to 20-25° C. and maintain the temperature for 2 hours. Add 4.9 g of hydroxybenzotriazole and 17.5 g of HBTU to the reaction mass. Next, 57 ml of diisopropylethylamine is added drop wise over a period of 15 minutes, and the reaction mass is stirred for 1 hour further. Again, 4.9 g of hydroxybenzotriazole and 17.5 g of HBTU are added. Next, 57 ml of diisopropylethylamine is added drop wise over a period of 15 minutes, and the reaction mass is stirred for 1 hour further. The progress of reaction is monitored by UPLC (the unreacted Fragment-(B+C) not more than 5%). In another container, 29750 ml of methanol is charged and added to the reaction at a temperature of 20-25° C. over a period of 10 minutes. Stir the reaction mass at a temperature of 20-25° C. over a period of 60 minutes. Filter the reaction mass under reduced pressure and wash the cake with a mixture of N,N-dimethyl formamide and methanol (1:5) 2100 ml solution. In another container, charge 5250 ml of water and 52.5 ml of hydrochloric acid. Add the cake into the solution and stir the mass for 30 minutes. Filter the reaction mass. Wash the cake with 5200 ml of water. In a separate container, charge 262.5 g of sodium bicarbonate and 5250 ml of water and stir to get a clear solution. Next, charge the cake into sodium bicarbonate solution and stir for 30 minutes. Stir the reaction mass for 30 minutes and filter the reaction mass. Further wash the cake with 5250 ml of methyl tertbutyl ether. Again, charge the cake into a 10 liter flask with 5250 ml of methyl tertbutyl ether. Stir the mass for 30 minutes and filter. Wash the cake with 5250 ml of methyl tertbutyl ether and suck dry. The cake is dried in a vacuum tray drier under reduced pressure at 45-50° C. for 15 hours to get a water content of not more than 6%.

Dry weight of Protected Plecanatide (SEQ ID NO: 3): 480 g

Yield w/w: 1.44 (Theory=1.84)

### Example 7: Preparation of Linear Plecanatide

Charge 1000 ml of TFA (trifluoroacetic acid) into 10 liter 4 neck round bottom flask. Charge TIPS (triisopropyl silane) 100 ml and 25 ml water. Cool the mixture to 0 to 10° C. Add 100 g of DTT (dithiothreitol) under stirring. Slowly add the 100 g of Protected Plecanatide (SEQ ID NO: 3) over a period for 5 min. Stir the reaction mass for 30 minutes at 0 to 10° C. Raise the temperature of reaction mass to 20-25° C. Stir the reaction mass for 1 hour 50 minutes at temp. 20-25° C. In another 10 liter round bottom flask charge 3500 ml of methyl tert.butyl ether and cool the mass to 0 to −10° C. Slowly add the TFA solution into cooled methyl tert.butyl ether over a period of 20 minutes at 0 to −10° C. The product precipitates out. Maintain the temperature of reaction mass at 0 to −10° C. for a further 45 minutes. Filter the reaction mass and wash the cake with 2×1000 ml methyl tert.butyl ether followed by washing with 500 ml of ethyl acetate. The wet cake is further slurry washed with 500 ml of acetonitrile. The product is isolated by filtration. Dry the product under reduced pressure at temperature 45-50° C.

Dry weight of Linear Plecanatide (SEQ ID NO: 9): 68 g

Yield w/w: 0.68

### Example 8: Preparation of Plecanatide

Charge 30 g of Linear Plecanatide (SEQ ID NO: 9) into a 20-liter round bottom flask fitted with an overhead stirrer. Charge purified water (13.5 L) and acetonitrile (600 ml) and maintain the temperature at 20-25° C. Adjust the pH with aq. ammonia (25%) to get the pH of the reaction mass to 8-9. Add 10.0 ml of 3.5% of hydrogen peroxide to the reaction mass and stir the reaction mass for 3 to 5 hours under air atmosphere. The progress of the reaction is monitored by HPLC (limit of unreacted mono cyclization to not more than 5%). After completion of monocyclization, the pH of reaction mass is adjusted to 3 to 4 by addition of acetic acid. Next, 12 g of iodine is dissolved into 400 ml of acetonitrile, and the iodine solution is added over a period of 15 minutes at 20-25° C. resulting in the reaction mass turning to a reddish-brown color. The dicyclization reaction is monitored by HPLC. When unreacted monocyclized is 5% NMT, the pH of reaction mixture is adjusted in between 6 to 7 by addition of aq. ammonia solution (25%), followed by addition of 120 g of Amberjet 9000-OH resin and stirred for 30 minutes. Filter the reaction mass to get a Crude Plecanatide (SEQ ID NO: 1) solution. The solution pH is adjusted to 4-5 with acetic acid and taken for purification by preparative HPLC. The first purification is performed using reverse phase preparative HPLC by using a 50 mm DAC Column packed with C18 RP silica and Mobile Phase A (0.05-0.10 M of ammonium acetate solution in water and acetic acid) and Mobile Phase B (75% Acetonitrile and 25% Mobile phase A. The product is eluted with 20% Mobile Phase B/Mobile Phase A to get the required Plecanatide fractions (purity >85%). These fractions are further pooled and diluted with 20% water. The diluted fractions are purified using Reverse phase preparative HPLC by using a 50 mm DAC Column packed with C18 silica with Mobile Phase A, 0.4-0.50% acetic acid in water and Mobile Phase B methanol. The product is eluted with 50% Mobile Phase B/Mobile Phase A to get the required Plecanatide fractions (purity >85%). These fractions are pooled and diluted with 20% water. The second purification is performed using Reverse phase preparative HPLC by using a 50 mm DAC Column packed with C18 silica and Mobile Phase A, 0.005-0.10 M Ammonium bicarbonate and Mobile Phase B (50% Acetonitrile and Mobile phase A. The above diluted fractions are injected, and the product is eluted with 20% Mobile phase B/Mobile phase A to get the required Plecanatide fractions (purity >97%). The pooled fractions having purity >97% are mixed together and pass through 300 Da membrane filtration to get about 70-80% by volume of concentrated mass. Finally, the concentrated mass is filtered through a 0.22 micron filter and lyophilized to get Plecanatide (Purity >97%).

Yield: 1.5 g.

Yield w/w: 0.03-0.07 (Based on input of Linear Plecanatide).

1. A process for purifying plecanatide, comprising;
providing a first peptide solution comprising a crude plecanatide, wherein the crude plecanatide has a sequence of SEQ ID NO: 1;
loading the first peptide solution onto a column compatible with reversed-phase high performance liquid chromatography (HPLC); and
eluting the purified plecanatide (SEQ ID NO: 1) in the first peptide solution loaded onto the DAC column to form a second peptide solution, wherein the elution is performed using a mobile phase comprising: (a) 20% of a first solution comprising 75% acetonitrile and 25% Mobile phase A, and (b) 80% of a second solution comprising 0.05-0.10 M ammonium acetate in water and acetic acid.

2. The process of claim 1, wherein the second peptide solution is eluted from the DAC column as a plurality of fractions, and the method further comprises:
diluting one or more of the plurality of fractions by 20% with water to form one or more diluted fractions;
loading one or more of the diluted fractions onto a DAC column, wherein the DAC column is packed with C18 reversed-phase silica; and
eluting the purified plecanatide (SEQ ID NO: 1) in the one or more diluted fractions loaded onto the DAC column to form a third peptide solution, wherein the elution is performed using a mobile phase comprising: (a) 50% of a first solution comprising methanol, and (b) 50% of a second solution comprising 0.4-0.5% acetic acid in water.

3. The process of claim 1, further comprising:
loading the second peptide solution onto a DAC column, wherein the DAC column is packed with C18 reversed-phase silica; and eluting the purified plecanatide (SEQ ID NO: 1) in the second peptide solution loaded onto the DAC column to form a third peptide solution, wherein the elution is performed using a mobile phase comprising: (a) 20% of a first solution comprising 50% acetonitrile and 50% Mobile Phase A, and (b) 80% of a second solution comprising 0.005-0.10 M ammonium bicarbonate.

4. The process of claim 3, wherein the third peptide solution is eluted from the DAC column as a plurality of fractions, and the method further comprises:
pooling at least two fractions selected from the plurality of fractions; and concentrating the pooled fractions using 250 to 400 Dalton membrane filtration to produce about 70-80% by volume of a concentrated mass.

5. The process of claim 2, further comprising:
loading the third peptide solution onto a DAC column, wherein the DAC column is packed with C18 reversed-phase silica; and
eluting the purified plecanatide (SEQ ID NO: 1) in the third peptide solution loaded onto the DAC column to form a fourth peptide solution, wherein the elution is performed using a mobile phase comprising: (a) 20% of a first solution comprising 50% acetonitrile and 50% Mobile Phase A, and (b) a second solution comprising 0.005-0.10 M ammonium bicarbonate.

6. The process of claim 5, wherein the fourth peptide solution is eluted from the DAC column as a plurality of fractions, and the method further comprises:
pooling at least two fractions selected from the plurality of fractions; and concentrating the pooled fractions using 250 to 400 Dalton membrane filtration to produce about 70-80% by volume of a concentrated mass.

7-21. (canceled)

22. The process of claim 4, wherein the concentrated mass is filtered and lyophilized to obtain the purified plecanatide (SEQ ID NO: 1) having a purity of greater than 98%.

23. The process of claim 6, wherein the concentrated mass is filtered and lyophilized to obtain the purified plecanatide (SEQ ID NO: 1) having a purity of greater than 98%.

24. The purified plecanatide of claim 22, wherein the purified plecanatide (SEQ ID NO: 1) is substantially free of one or more of the following impurities:
a) a Plecanatide topoisomer impurity represented by the following:
L-Leucine, L-asparaginyl-L-alpha-aspartyl-L-alpha-glutamyl-L-cysteinyl-L-alpha-glutamyl-L-leucyl-L-cysteinyl-L-valyl-L-asparaginyl-L-valyl-L-alanyl-L-cysteinyl-L-threonylglycyl-L-cysteinyl-, Cyclic (4→12),(7→15)-bis(disulfide), 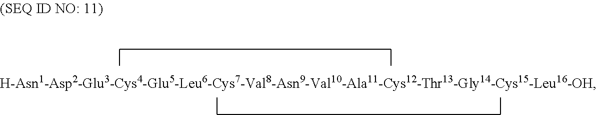
b) a 9-Asp-Plecanatide impurity represented by the following:
L-Leucine, L-asparaginyl-L-alpha-aspartyl-L-alpha-glutamyl-L-cysteinyl-L-alpha-glutamyl-L-leucyl-L-cysteinyl-L-valyl-L-aspartyl-L-valyl-L-alanyl-L-cysteinyl-L-threonylglycyl-Lcysteinyl-, Cyclic (4→12),(7→15)-bis(disulfide), 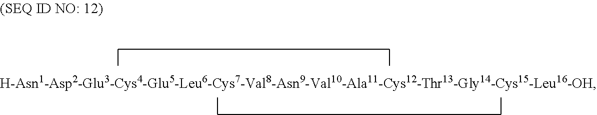
c) a 2-Iso-Asp-Plecanatide impurity represented by the following:
L-Leucine, L-asparaginyl-L-alpha-iso-aspartyl-L-alpha-glutamyl-L-cysteinyl-L-alpha-glutamyl-L-leucyl-L-cysteinyl-L-valyl-L-asparaginyl-L-valyl-L-alanyl-L-cysteinyl-L-threonylglycyl-L-cysteinyl-, Cyclic (4→12),(7→15)-bis(disulfide), 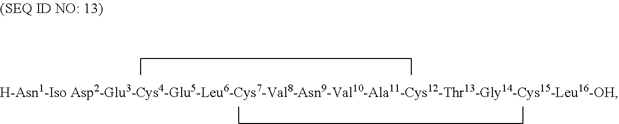
d) a Plecanatide Isomer-I impurity represented by the following:
L-Leucine, L-asparaginyl-L-alpha-aspartyl-L-alpha-glutamyl-L-cysteinyl-L-alpha-glutamyl-L-leucyl-L-cysteinyl-L-valyl-L-asparaginyl-L-valyl-L-alanyl-L-cysteinyl-L-threonylglycyl-L-cysteinyl-, Cyclic (4→7),(12→15)-bis(disulfide), 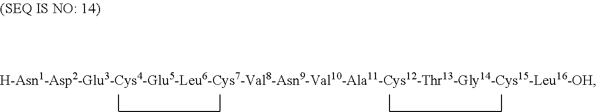
and
e) a Plecanatide isomer-II impurity represented by the following:
L-Leucine, L-asparaginyl-L-alpha-aspartyl-L-alpha-glutamyl-L-cysteinyl-L-alpha-glutamyl-L-leucyl-L-cysteinyl-L-valyl-L-asparaginyl-L-valyl-L-alanyl-L-cysteinyl-L-threonylglycyl-L-cysteinyl-, Cyclic (4→15),(7→12)-bis(disulfide).

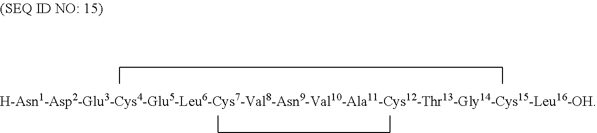

25. The purified plecanatide of claim 23, wherein the purified plecanatide (SEQ ID NO: 1) is substantially free of one or more of the following impurities:
a) a Plecanatide topoisomer impurity represented by the following:
L-Leucine, L-asparaginyl-L-alpha-aspartyl-L-alpha-glutamyl-L-cysteinyl-L-alpha-glutamyl-L-leucyl-L-cysteinyl-L-valyl-L-asparaginyl-L-valyl-L-alanyl-L-cysteinyl-L-threonylglycyl-L-cysteinyl-, Cyclic (4→12),(7→15)-bis(disulfide), 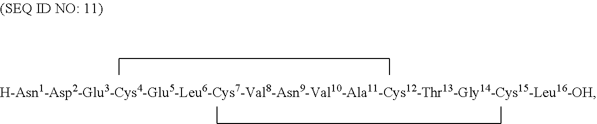
b) a 9-Asp-Plecanatide impurity represented by the following:
L-Leucine, L-asparaginyl-L-alpha-aspartyl-L-alpha-glutamyl-L-cysteinyl-L-alpha-glutamyl-L-leucyl-L-cysteinyl-L-valyl-L-aspartyl-L-valyl-L-alanyl-L-cysteinyl-L-threonylglycyl-Lcysteinyl-, Cyclic (4→12), (7→15)-bis(disulfide), 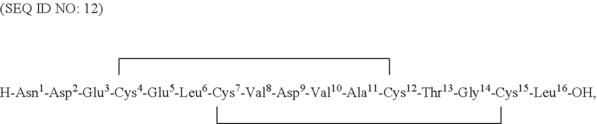
c) a 2-Iso-Asp-Plecanatide impurity represented by the following:
L-Leucine, L-asparaginyl-L-alpha-iso-aspartyl-L-alpha-glutamyl-L-cysteinyl-L-alpha-glutamyl-L-leucyl-L-cysteinyl-L-valyl-L-asparaginyl-L-valyl-L-alanyl-L-cysteinyl-L-threonylglycyl-L-cysteinyl-, Cyclic (4→12),(7→15)-bis(disulfide), 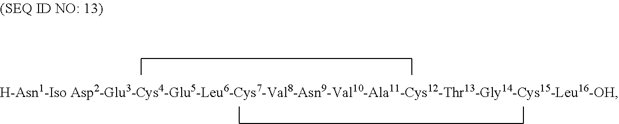
d) a Plecanatide Isomer-I impurity represented by the following:
L-Leucine, L-asparaginyl-L-alpha-aspartyl-L-alpha-glutamyl-L-cysteinyl-L-alpha-glutamyl-L-leucyl-L-cysteinyl-L-valyl-L-asparaginyl-L-valyl-L-alanyl-L-cysteinyl-L-threonylglycyl-L-cysteinyl-, Cyclic (4→7),(12→15)-bis(disulfide), 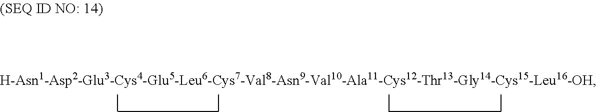
and
e) a Plecanatide isomer-II impurity represented by the following:
L-Leucine, L-asparaginyl-L-alpha-aspartyl-L-alpha-glutamyl-L-cysteinyl-L-alpha-glutamyl-L-leucyl-L-cysteinyl-L-valyl-L-asparaginyl-L-valyl-L-alanyl-L-cysteinyl-L-threonylglycyl-L-cysteinyl-, Cyclic (4→15),(7→12)-bis(disulfide).

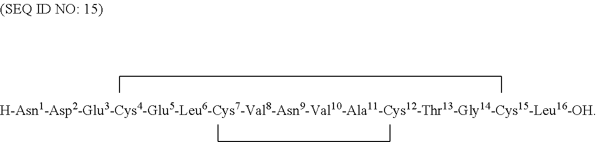

26. A process for the preparation of a plecanatide comprising:
a first intermediate having a structure represented by the following:
(SEQ ID NO: 2)
Boc-Asn-Asp(OtBU)-Glu(OtBu)-Cys(Trt)-
Glu(OtBu)-Leu-OH;
a second intermediate having a structure represented by the following:
(SEQ ID NO: 8)
H-Cys(Acm)-Val-Asn(Trt)-Val-Ala-
Cys(Trt)-Thr(tBu)-Gly-Cys(Acm)-
Leu-OtBu;
and
coupling the first and the second intermediates to yield a protected plecanatide represented by the following:
(SEQ ID NO: 3)
Boc-Asn-Asp(OtBu)-Glu(OtBu)-
Cys(Trt)-Glu(OtBu)-Leu-Cys
(Acm)-Val-Asn(Trt)-Val-Ala-
Cys(Trt)-Thr(tBu)-Gly-Cys(Acm)-
Leu-OtBu.

27. The process of claim 26, further comprising cleaving the protected plecanatide (SEQ ID NO: 3) by using reducing or deprotecting agents to obtain linear plecanatide (SEQ ID NO: 9).

28. The process of claim 27, wherein the reducing or deprotecting agents are triisopropyl silane (TIPS) with trifluoro acetic acid in the presence of dithiothreitol (DTT).

29. A partially-protected compound selected from the group consisting of:
(SEQ ID NO: 2)
Boc-Asn-Asp(OtBU)-Glu(OtBu)-Cys(Trt)-
Glu(OtBu)-Leu-OH
and
(SEQ ID NO: 3)
Boc-Asn-Asp(OtBu)-Glu(OtBu)-Cys(Trt)-
Glu(OtBu)-Leu-Cys(Acm)-Val-Asn(Trt)-
Val-Ala-Cys(Trt)-Thr(tBu)-Gly-
Cys(Acm)-Leu-OtBu.


SEQUENCE LISTING (15 sequences)

SEQ ID NO: 1 - protein, 16 aa, Artificial Sequence
  Plecanatide
  DISULFID (4)..(12)
  DISULFID (7)..(15)
NDECELCVNVACTGCL

SEQ ID NO: 2 - protein, 6 aa, Artificial Sequence
  Fragment A
  MISC_FEATURE (1)..(1): tert-butyloxycarbonyl
  MISC_FEATURE (2)..(2): tert-butyl
  MISC_FEATURE (3)..(3): tert-butyl
  MISC_FEATURE (4)..(4): trityl
  MISC_FEATURE (5)..(5): tert-butyl
NDECEL

SEQ ID NO: 3 - protein, 16 aa, Artificial Sequence
  Protected Plecanatide
  MISC_FEATURE (1)..(1): tert-butyloxycarbonyl
  MISC_FEATURE (2)..(2): tert-butyl
  MISC_FEATURE (3)..(3): tert-butyl
  MISC_FEATURE (4)..(4): trityl
  MISC_FEATURE (5)..(5): tert-butyl
  MISC_FEATURE (7)..(7): acetamidomethyl
  MISC_FEATURE (9)..(9): trityl
  MISC_FEATURE (12)..(12): trityl
  MISC_FEATURE (13)..(13): tert-butyl
  MISC_FEATURE (15)..(15): acetamidomethyl
  MISC_FEATURE (16)..(16): tert-butyl
NDECELCVNVACTGCL

SEQ ID NO: 4 - protein, 6 aa, Artificial Sequence
  Fragment A Resin
  MISC_FEATURE (1)..(1): tert-butyloxycarbonyl
  MISC_FEATURE (2)..(2): tert-butyl
  MISC_FEATURE (3)..(3): tert-butyl
  MISC_FEATURE (4)..(4): trityl
  MISC_FEATURE (5)..(5): tert-butyl
  MISC_FEATURE (6)..(6): chlorotrityl chloride resin
NDECEL

SEQ ID NO: 5 - protein, 8 aa, Artificial Sequence
  Fragment B Resin
  MISC_FEATURE (1)..(1): fluorenylmethoxycarbonyl
  MISC_FEATURE (1)..(1): acetamidomethyl
  MISC_FEATURE (3)..(3): trityl
  MISC_FEATURE (6)..(6): trityl
  MISC_FEATURE (7)..(7): tert-butyl
  MISC_FEATURE (8)..(8): chlorotrityl chloride resin
CVNVACTG

SEQ ID NO: 6 - protein, 8 aa, Artificial Sequence
  Fragment B
  MISC_FEATURE (1)..(1): fluorenylmethoxycarbonyl
  MISC_FEATURE (1)..(1): acetamidomethyl
  MISC_FEATURE (3)..(3): trityl
  MISC_FEATURE (6)..(6): trityl
  MISC_FEATURE (7)..(7): tert-butyl
CVNVACTG

SEQ ID NO: 7 - protein, 10 aa, Artificial Sequence
  Protected Fragment B+C
  MISC_FEATURE (1)..(1): fluorenylmethoxycarbonyl
  MISC_FEATURE (1)..(1): acetamidomethyl
  MISC_FEATURE (3)..(3): trityl
  MISC_FEATURE (6)..(6): trityl
  MISC_FEATURE (7)..(7): tert-butyl
  MISC_FEATURE (9)..(9): acetamidomethyl
  MISC_FEATURE (10)..(10): tert-butyl
CVNVACTGCL

SEQ ID NO: 8 - protein, 10 aa, Artificial Sequence
  Fragment B+C
  MISC_FEATURE (1)..(1): acetamidomethyl
  MISC_FEATURE (3)..(3): trityl
  MISC_FEATURE (6)..(6): trityl
  MISC_FEATURE (7)..(7): tert-butyl
  MISC_FEATURE (9)..(9): acetamidomethyl
  MISC_FEATURE (10)..(10): tert-butyl
CVNVACTGCL

SEQ ID NO: 9 - protein, 16 aa, Artificial Sequence
  Linear Plecanatide
  MISC_FEATURE (7)..(7): acetamidomethyl
  MISC_FEATURE (15)..(15): acetamidomethyl
NDECELCVNVACTGCL

SEQ ID NO: 10 - protein, 16 aa, Artificial Sequence
  Mono Cyclized Plecanatide
  DISULFID (4)..(12)
  MISC_FEATURE (7)..(7): acetamidomethyl
  MISC_FEATURE (15)..(15): acetamidomethyl
NDECELCVNVACTGCL

SEQ ID NO: 11 - protein, 16 aa, Artificial Sequence
  Topoisomer Plecanatide
  DISULFID (4)..(12)
  DISULFID (7)..(15)
NDECELCVNVACTGCL

SEQ ID NO: 12 - protein, 16 aa, Artificial Sequence
  9-Asp-Plecanatide
  DISULFID (4)..(12)
  DISULFID (7)..(15)
NDECELCVDVACTGCL

SEQ ID NO: 13 - protein, 16 aa, Artificial Sequence
  2-Iso-Asp-Plecanatide
  MISC_FEATURE (2)..(2): isoaspartic acid
  DISULFID (4)..(12)
  DISULFID (7)..(15)
NDECELCVNVACTGCL

SEQ ID NO: 14 - protein, 16 aa, Artificial Sequence
  Plecanatide Isomer-I
  DISULFID (4)..(7)
  DISULFID (12)..(15)
NDECELCVNVACTGCL

SEQ ID NO: 15 - protein, 16 aa, Artificial Sequence
  Plecanatide Isomer-II
  DISULFID (4)..(15)
  DISULFID (7)..(12)
NDECELCVNVACTGCL